(19) United States
(12) Patent Application Publication
(10) Pub. No.: US 2016/0296500 A1
(43) Pub. Date: Oct. 13, 2016

(54) TACROLIMUS FOR USE IN TREATING DISEASES CHARACTERIZED BY PROTEIN AGGREGATE DEPOSITION IN NEURONAL CELLS

(71) Applicant: CHRONOS THERAPEUTICS LIMITED, Oxford (GB)

(72) Inventors: Alexandre AKOULITCHEV, Ewelme, Oxfordshire (GB); Elizabeth MILWAY, Oxford, Oxfordshire (GB); Elizabeth Jane MELLOR, Kennington, Oxford, Oxfordshire (GB); Michael YOUDELL, Oxford, Oxfordshire (GB)

(21) Appl. No.: 14/893,181

(22) PCT Filed: May 22, 2014
(86) PCT No.: PCT/GB2014/051570

§ 371 Date: Nov. 23, 2015

(30) Foreign Application Priority Data
May 24, 2013 (GB) 1309375.2

(51) Int. Cl. A61K 31/436; A61K 9/00

(52) U.S. Cl. CPC A61K 31/436; A61K 9/0053

(57) ABSTRACT

This invention relates to the use of a very low dose of tacrolimus or a close structural analogue to treat a disease characterised by deposition of protein aggregates in neuronal cells. More particularly this invention relates to the use of a very low dose of tacrolimus for the treatment of amyotrophic lateral sclerosis.

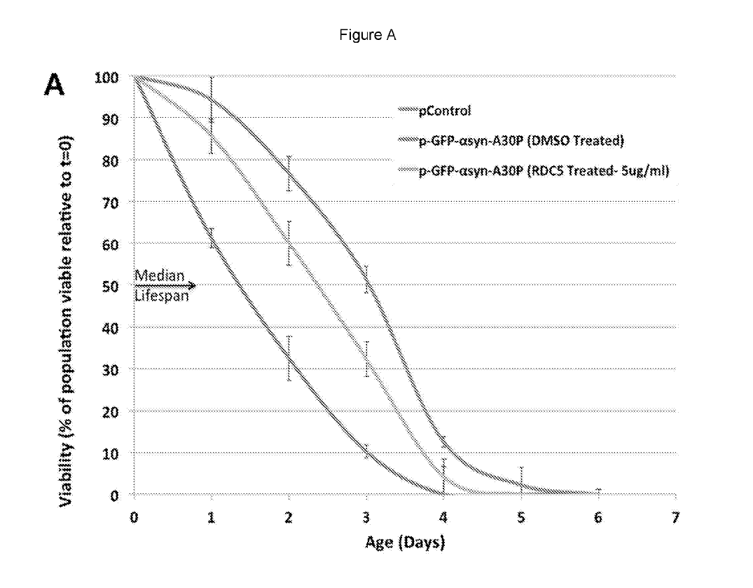

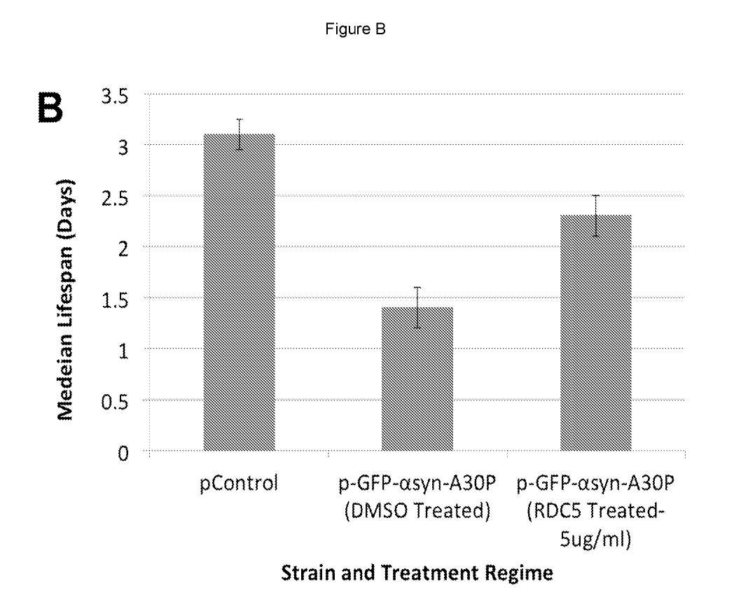

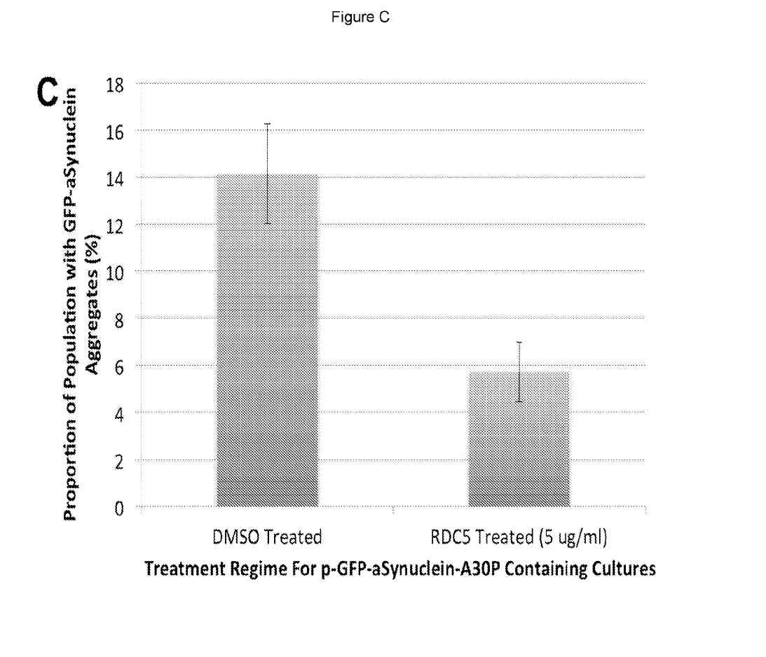

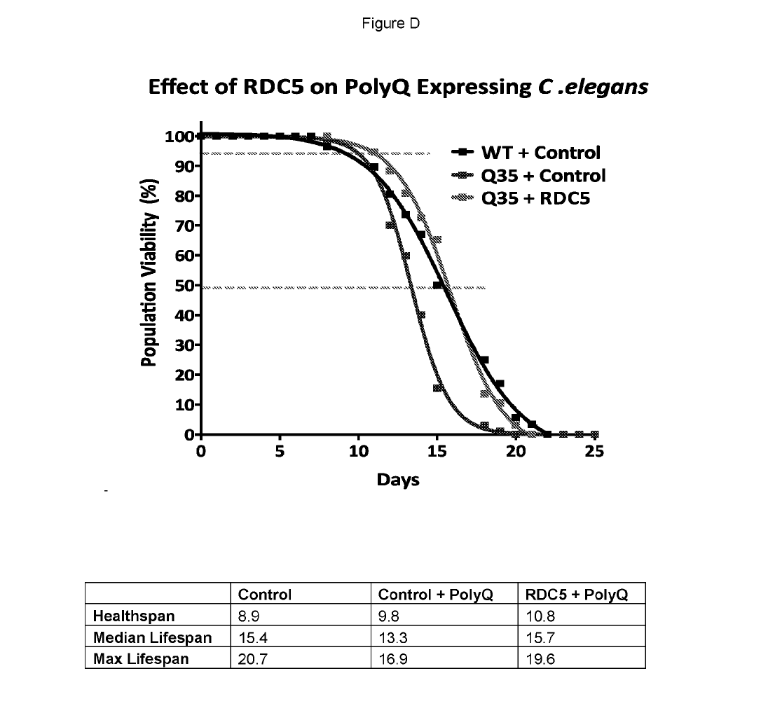

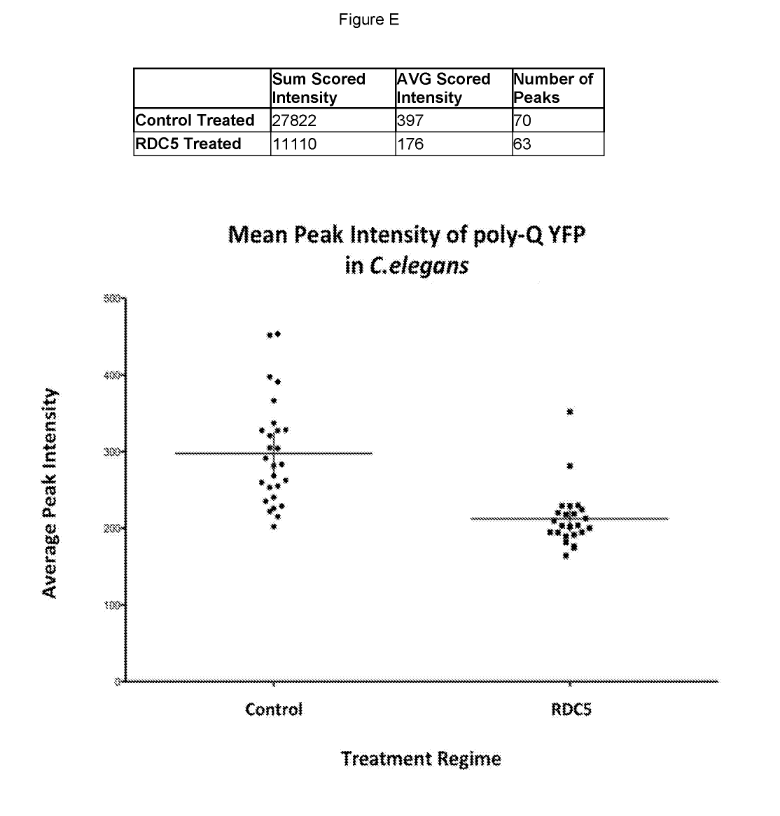

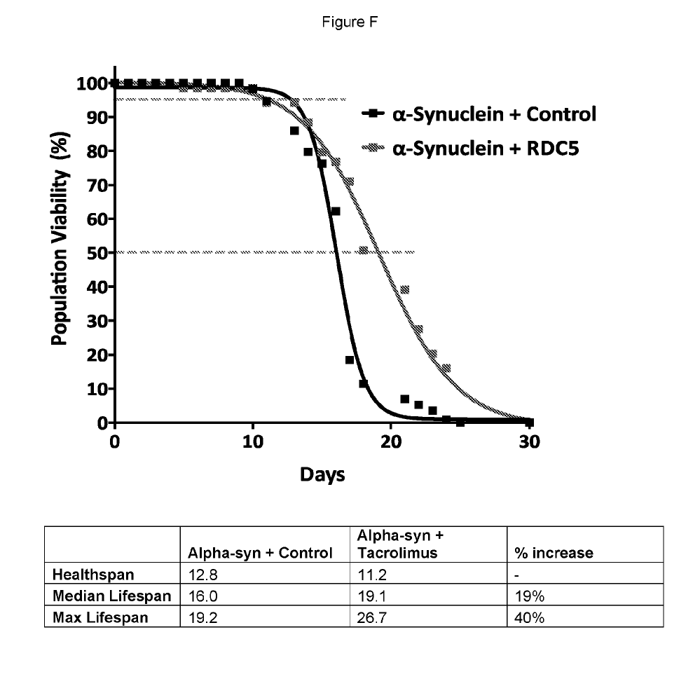

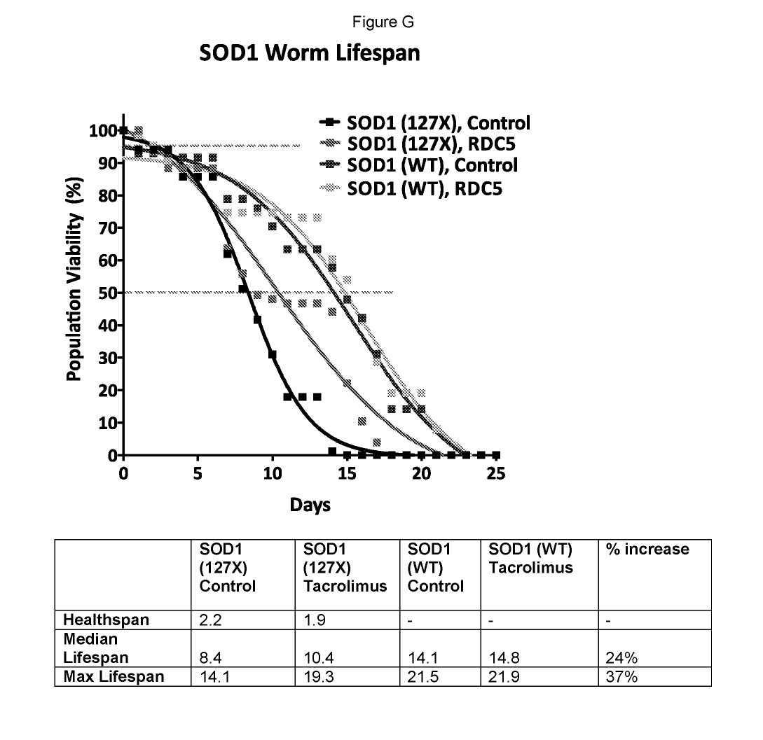

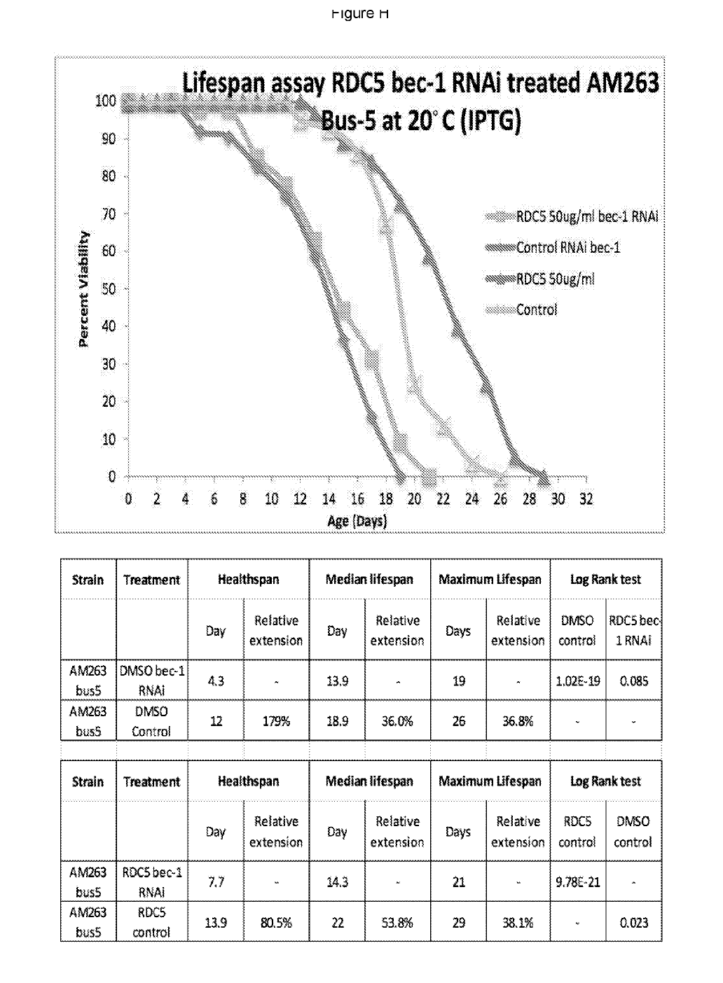

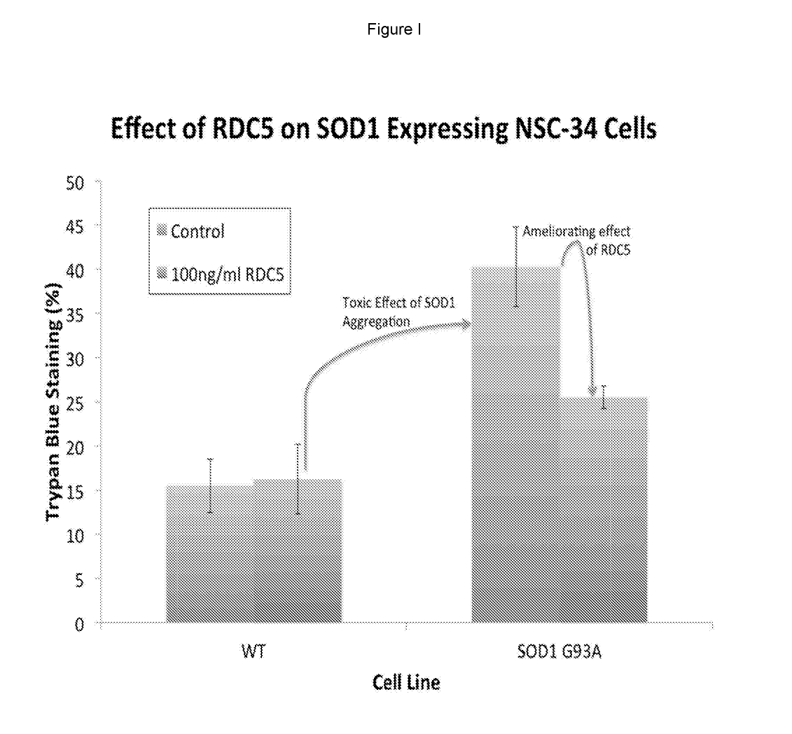

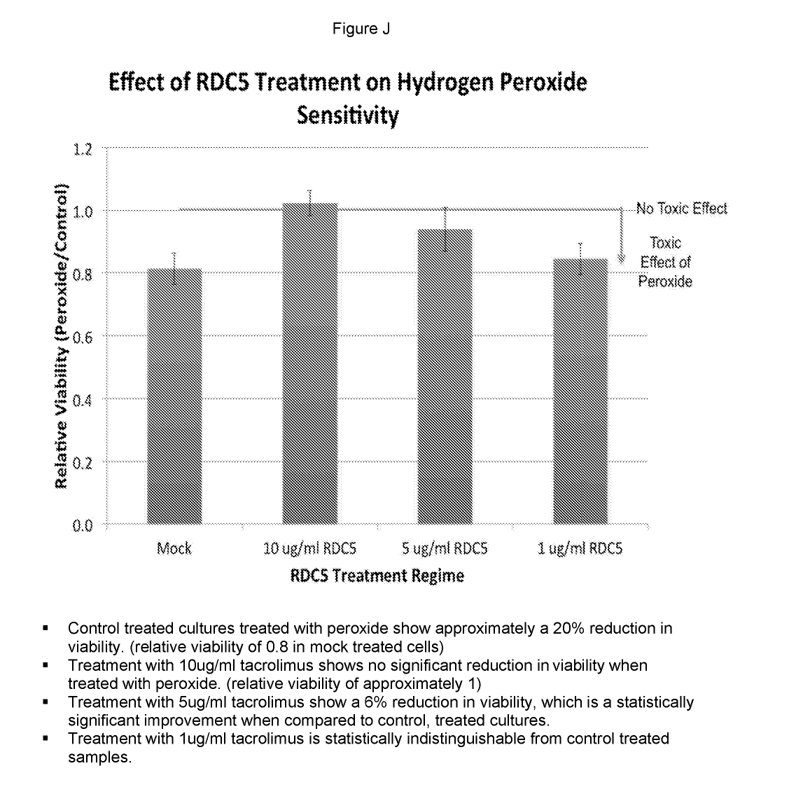

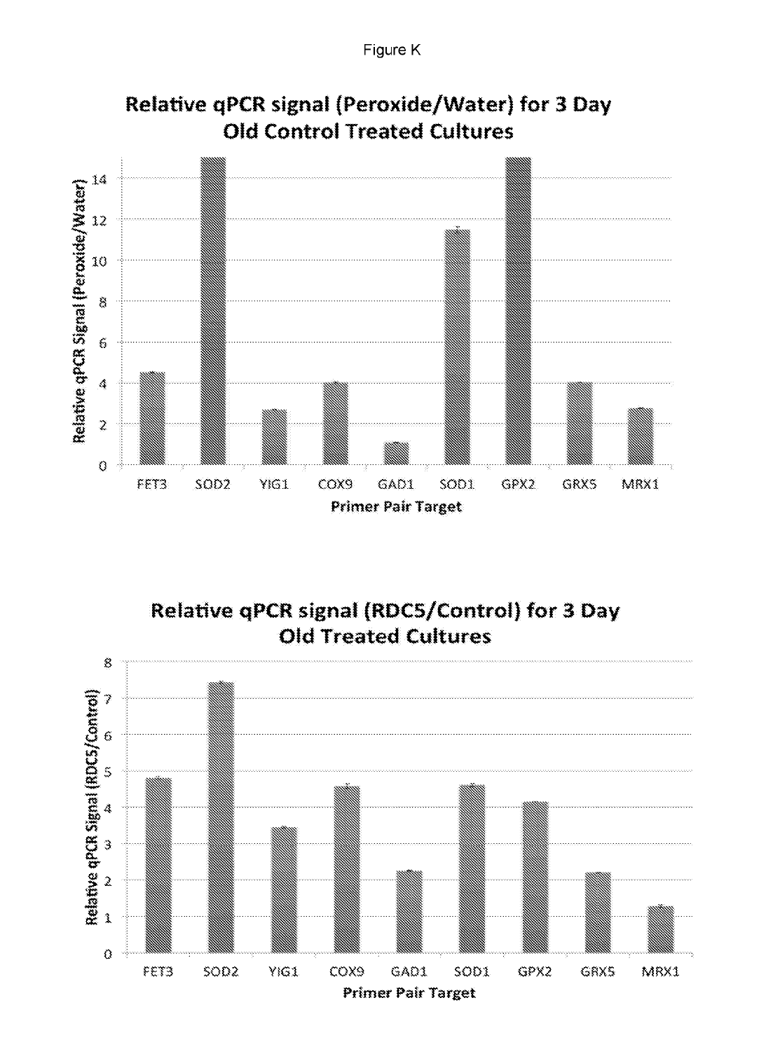

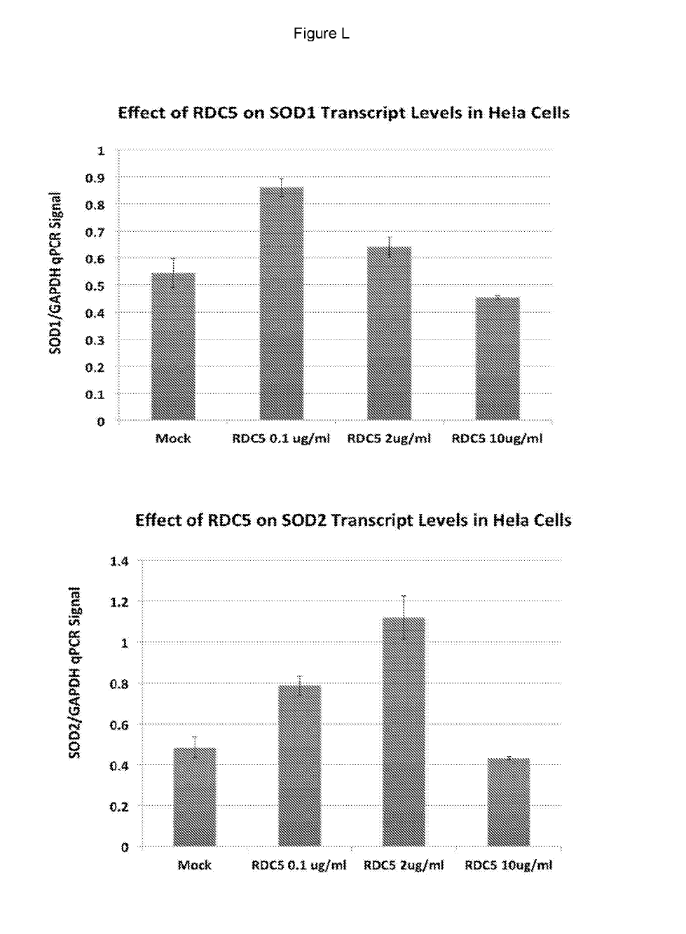

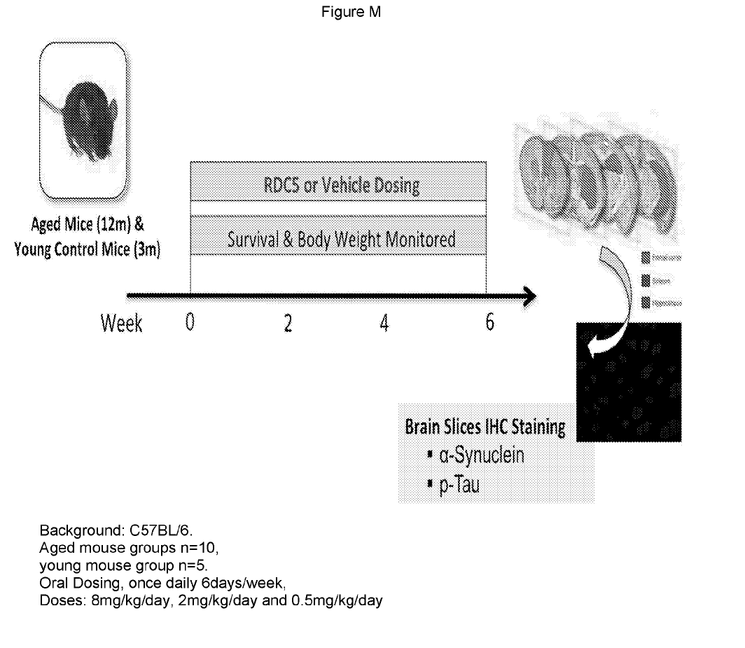

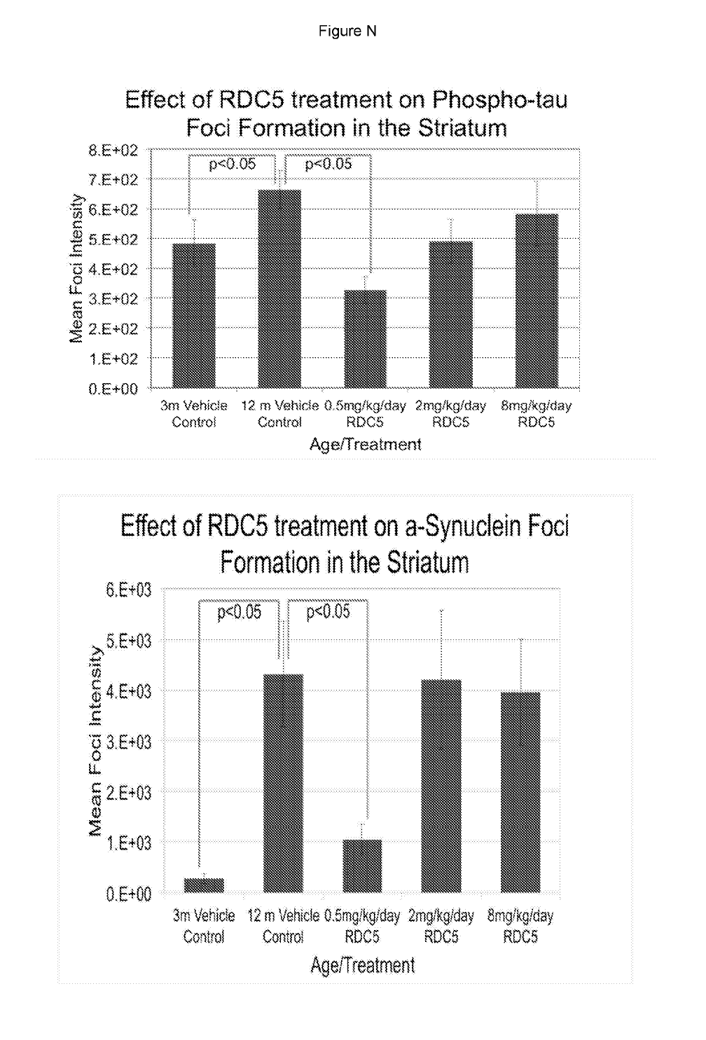

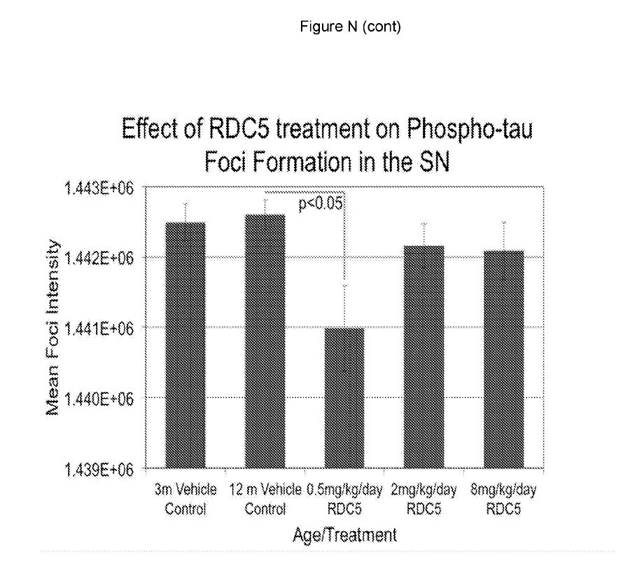

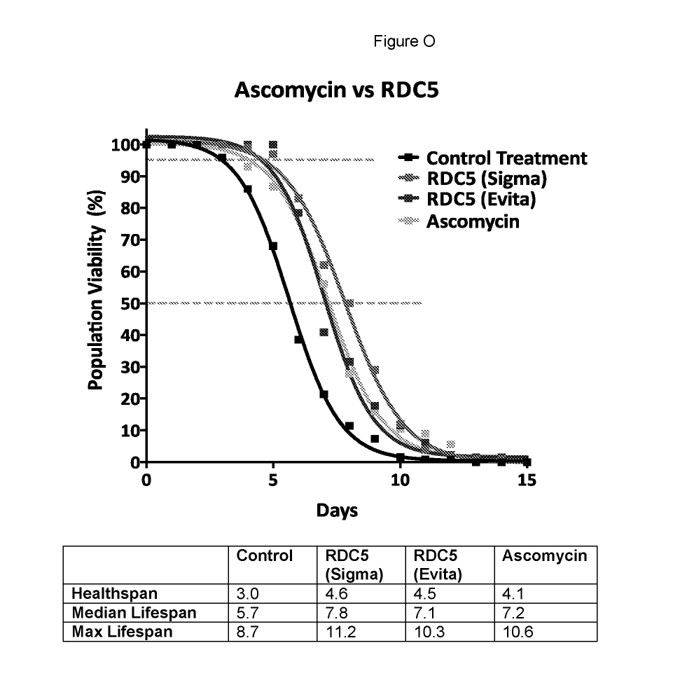

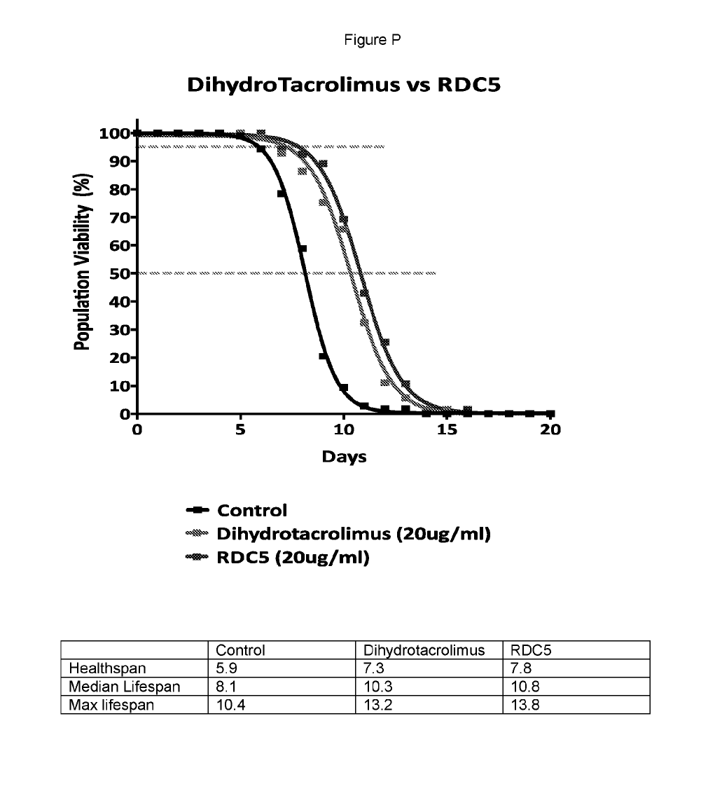

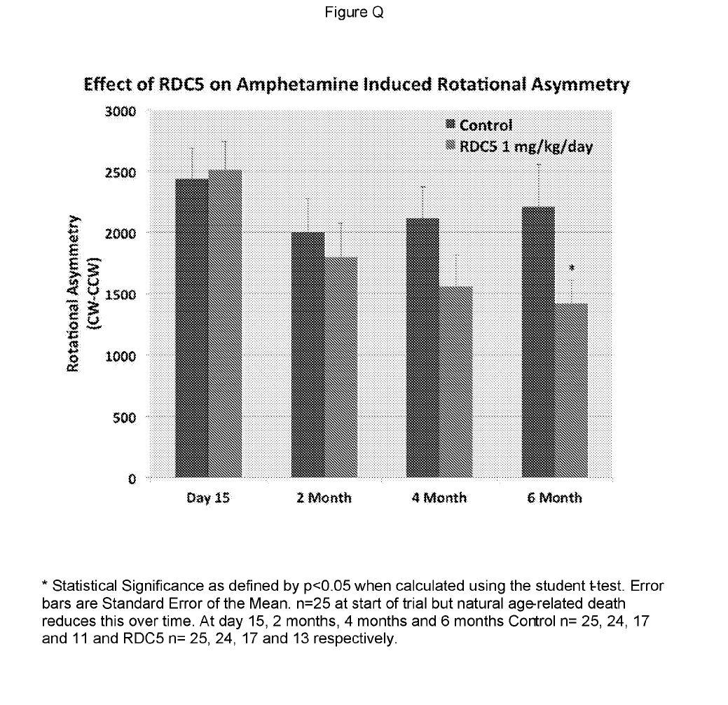

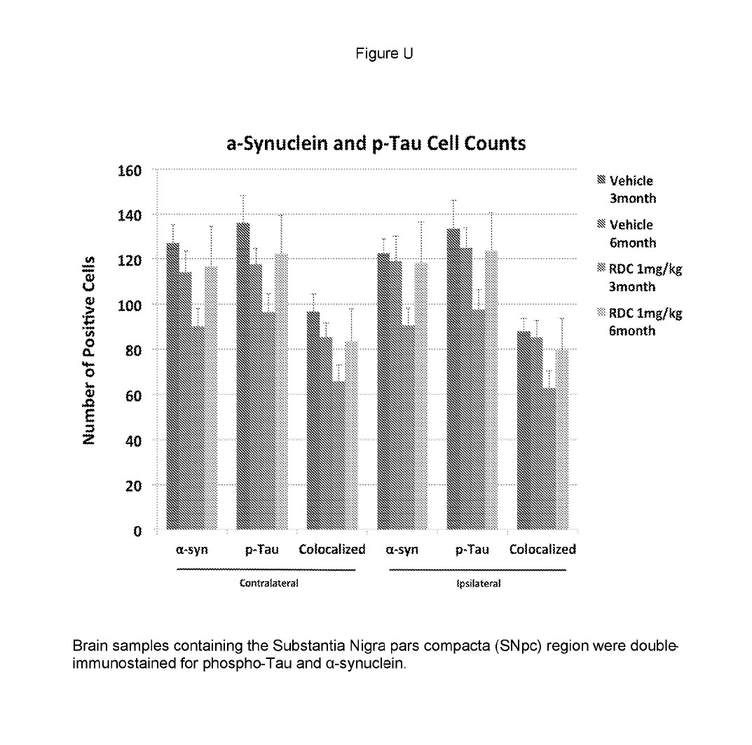

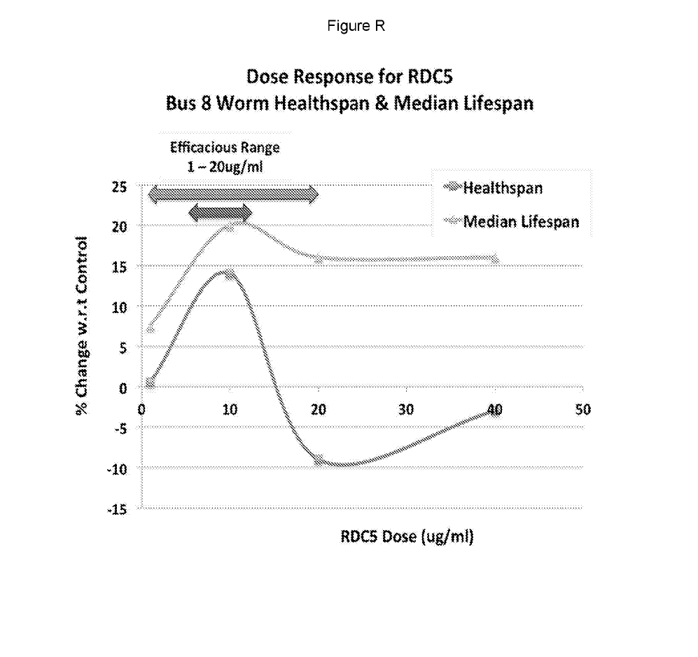

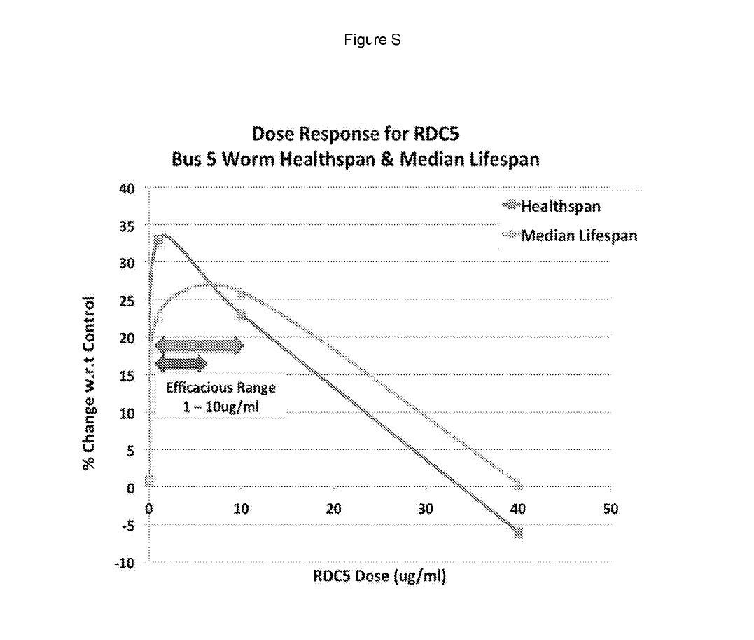

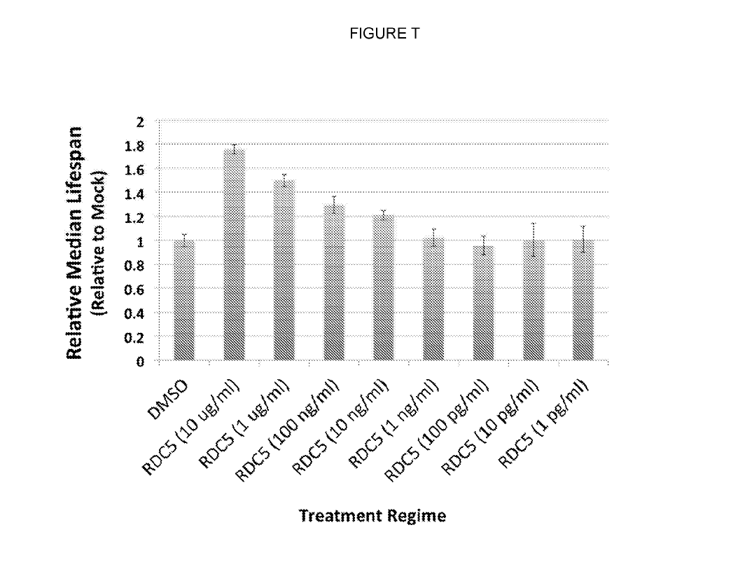

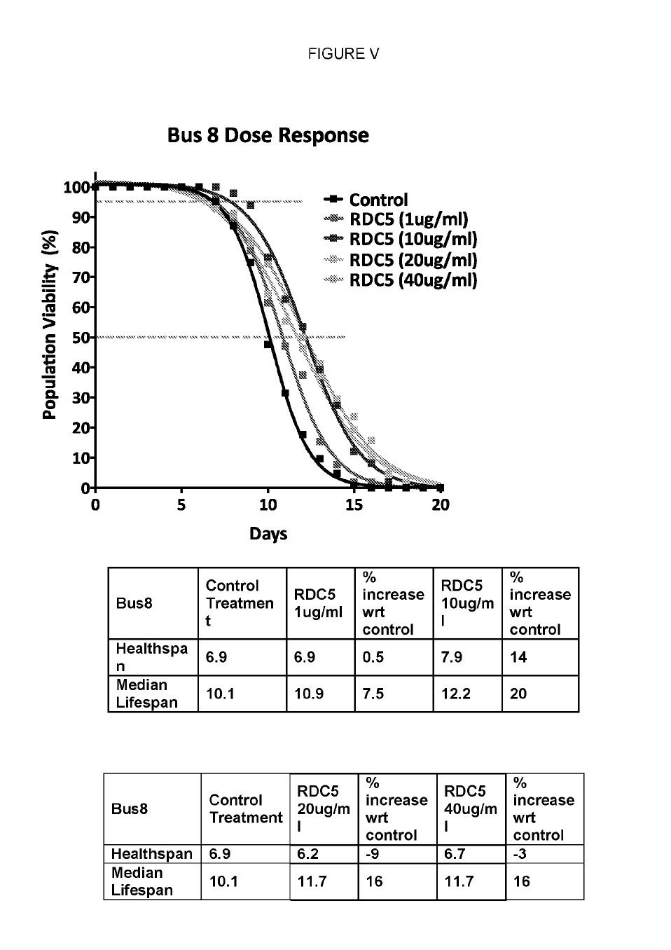

This invention relates to the use of a very low dose of tacrolimus or a close structural analogue to treat a disease characterised by deposition of protein aggregates in neuronal cells. More particularly this invention relates to the use of a very low dose of tacrolimus for the treatment of amyotrophic lateral sclerosis.

Tacrolimus (also called fujimycin or FK506) is clinically employed as an immunosuppressant, for example, in patients who have had organ transplants, and for the treatment of ulcerative colitis or certain skin conditions. Tacrolimus is available under trade names such as Prograf®, Advagraf® and Protopic®. Commercially available dosage forms of tacrolimus include capsules containing 0.5 mg, 1 mg, 3 mg and 5 mg and ointments for skin conditions where the concentration is 0.05% to 0.19%. Tacrolimus is most commonly administered twice a day for immunosuppression to prevent rejection of transplanted tissues. The clinically employed dose is generally adjusted to produce a whole blood trough concentration of at least 4 mg/mL when seeking to prevent rejection. This is achieved by employing a recommended initial oral dose (two divided doses every 12 hours) which is in the range 0.075 mg/kg/day to 0.2 mg/kg/day which for an average 70 kg patient required two daily doses of about 2.5 mg to 14 mg. Tacrolimus has also been employed for the treatment of arthritis generally at 3 mg per day. Possibly the lowest dose employed for routine clinical immunosuppression was recorded was for the treatment of myasthenia gravis was 2-3 mg per day (Kanshi et al., J. Neurol. Neurosurg. Psychiatry 2005; 76: 448-450) but this was together with up to 50 mgs per day of prednisolone (and was administered to a lady who may have had low body weight). Clinical trials using low doses of tacrolimus in treating rheumatoid arthritis were described in an editorial in Rheumatology, 2004, 43; 946-948 where in phase II doses of 1 mg, 3 mg and 5 mg of tacrolimus per day were employed and in view of the data from the phase II study, a phase III study was performed using 2 mg and 3 mg per day of tacrolimus.

It is believed that these established clinical uses for tacrolimus operate via a mechanism which acts via calmodulin to activate calcineurin which thus inhibits both T-lymphocyte signal transduction and IL-2 transcription. These are dose dependent mechanism so that greater the amount of tacrolimus administered the greater the immunosuppression. This mechanism is not involved in the present invention which is fortunate because immunosuppression is desirable when treating the conditions set forth above, but since it leads to a reduction in immunovigilance, it is not without its disadvantages such as increased risk of infection or lymphoma.

WO 2011/004194 discloses that tacrolimus may be used for the treatment of certain disorders. However, WO 2011/004194 did not disclose that a dose different from conventional immunosuppressant doses of tacrolimus should be employed to treat such diseases.

WO 2000/15208 discloses that tacrolimus may be used for the treatment of certain diseases and notes that the daily dose for chronic use is from 0.1 mg/kg to 30 mg/kg so that for a 70 kg person the daily dose would be 7.5 mg to 210 mg. This range is at least as high as the normal dose range suggested for use of tacrolimus as an immunosuppressant. U.S. 2004/007767 relates to the use of a modified tacrolimus having a methyl group at C₂₁ instead of the propenyl group present in tacrolimus and this also discloses that the daily dose for chronic use is from 0.1 mg/kg to 30 mg/kg. Gerard et al., J. Neurosciences, 2010, 30(7): 2454-2463 noted that immunophilin ligands including tacrolimus may exhibit neuroprotective effects via inhibition of FKBPs and that the observations validated FKBPs a novel drug targets for Parkinson's disease. Work by others undertaken to develop non-immunosuppressive immunophilin ligands (thereby avoiding undesired effects) was reference and it was noted that GP1-1485, one of such non-immunosuppressive analogues of tacrolimus, did not benefit patients with Parkinson's disease.

WO 2010/056754 disclosed microcapsulated inhibitors of mTOR, especially rapamycin, which could be used for a range of age related disorders. Individual doses were disclosed which were within the range 0.001 mg to 100 mg or even higher and particularised dose ranges of 5 mg/kg to 100 mg/kg were noted. Only effects of rapamycin were exemplified.

Pong et al., Current Drug Targets, 2003, 2: 349-356, discloses that immunophilia ligands may be considered for the treatment of neurodegenerative diseases. It notes that acrolimus can inhibit calcineurin and that the mechanism of action of non-immunosuppressive ligands in neuroprotection are unknown. It however noted that attempts had been made to move away from immunogenic molecules and to provide non-immunosuppressant ligands of different structures for use. It further noted that tacrolimus had been shown to be ineffective in the treatment of ALS.

Chattapadhanga et al., Current Medicinal Chemistry, 2011, 18: 5380-5397 discusses the role of neuroimmunophilia ligands and refers to tacrolimus and its C₂₁ ethyl and C₁₈ hydroxyl analogues as first generation ligands. It then describes how the skilled person has moved on to second and third generation ligands in the hope of finding effective medicaments for neurodegenerative disorders.

U.S. 2010/0081681 and U.S. 2013/0102569 disclose that inhibitors of TOR such as rapamycin and analogues may be used to inhibit age related diseases and mentions the immunosuppressive effects of rapamycin, cyclosporine A and tacrolimus. The experimental data was limited to rapamycin and no suggestion was made that doses could be employed in therapy which were less than immunosuppressant doses.

It has surprisingly been discovered that very lost doses of tacrolimus can provide benefit in the treatment of ALS at very low levels because it acts through an epigenic mechanism by which it modulates certain gene activity, for example switches on or upregulates certain genes, for example density to resistance to oxidative stress.

The general toxic effects of tacrolimus are believed to be related to its immunosuppressant mechanism (Dumant et al., J. Exp. Med. 1992, 176: 751-760) so that use of a dose less than that which results in immunosuppression similarly obviates general toxicity. The reduction in oxidative stress by use of the very low doses of tacrolimus or close structural analogue thereof is advantageous since oxidantive stress is believed to be a factor in many neurodegenerative conditions (Smith et al., Neurochemistry International, 2013, 62: 764-775). This has benefits in avoiding side effects such those mentioned above and other side effects such as hyperglycaemia and type II diabetes.

Amyotrophic lateral sclerosis (ALS) is a fatal neurodegenerative disease that causes progressive paralysis due to motor neurone death particularly in the motor cortex, spine and brain stem. The disease is often fatal within three years due to atrophy of muscles required for breathing, for example of the diaphragm. Often ALS has a focus in the spinal or bulbar regions of the central nervous system where loss of motor neurons is most pronounced and the loss of motor neurons tends to diminish with distance from that site. A considerable body of evidence suggests that oxidases such as superoxide dismutase 1 (SOD1), possibly through mutant forms thereof, plays a role in the development of the disease. It appears that aggregates form as a result of oxidative stress and this is accompanied by cell dysfunction or death. No treatments are available in routine clinical use that slow or reverse the progression or disease.

It has now been surprisingly found that it is possible to control expression of oxidases such as SOD1 and/or SOD2 epigenetically by the use of the correct very low dose of tacrolimus or a close structural analogue thereof. It has further been surprisingly found that it has a beneficial effect on mechanisms involved in neuronal damage resulting from oxidative stress in that expression of oxidative enzymes are normalised (reducing damage) and that mechanisms involved in enhancing cell health are increased (for example, by enhancing autophagy).

Although this is of most immediate use in respect of ALS, it is believed that it will also be of benefit for treatment of other diseases where cell damage is associated for the formation of protein aggregates.

The dose of tacrolimus or a close structural analogue thereof employed is less than that used to produce its clinical immunosuppressant effects when treating organ rejection or diseases such as arthritis or myasthenia gravis.

The use of tacrolimus for the treatment purposes described herein is presently preferred over that of a close structural analogue.

The mechanism by which tacrolimus or a close structural analogue provides it desirable effect in the present invention is not related to the mechanism by which it achieves immunosuppression. Indeed, use at conventional immunosuppressant doses are believed to be at best poorly ineffective. Tacrolimus and its close structural analogues have now been found to have a dose response curve with a pronounced bell shape with an upper cut off well below conventional immunosuppressant doses.

### BRIEF SUMMARY OF INVENTION

The present invention provides a method of treating a disease characterised by protein aggregate deposition in neuronal cells which comprises administering to a human in need thereof not more than once a day an effective amount of tacrolimus or a close structural analogue thereof in a dose which does not cause immunosuppression and which produces a trough whole blood level of tacrolimus or its close structural analogue of at least 0.05 ng/m L.

Apt diseases for treatment by such a method include in particular ALS, Parkinson's disease, Alzheimer's disease and Huntington's disease.

Suitably the method described herein comprises administering not more than once a day a dose of 0.001 mg/kg to 0.02 mg/kg of tacrolimus or a close structural analogue thereof.

The methods described herein may lead to an increase in autophagy and/or an improvement in oxidative stress and may involve epigenetic modification.

### DESCRIPTION OF THE FIGURES

Figure A shows the effect of tacrolimus on median lifespan in yeast expressing alpha-synuclein.

Figure B shows the time taken in yeast to reach 50% of their starting viability when treated with tacrolimus.

Figure C shows the effect of tacrolimus in the proportion of yeast cells with visible p-GFP-asyn-A30P aggregates.

Figure D shows the effect of tacrolimus on the viability of worms (C. elegans).

Figure E shows the effect of tacrolimus on the formulation of poly Q-YFP in worms (C. elegans).

Figure F shows the effect of tacrolimus on the median lifespan of worms expressing potentially neurotoxic alpha-synclein.

Figure G shows the effect of tacrolimus on the median and maximum lifespan of worms expressing toxic aggregates of SOD1.

Figure H shows the effect of tacrolimus on the median and maximum lifespans on worms expressing SOD1 mutant AM263.

Figure I shows the effect of tacrolimus in mammalian cells expressing SOD1-G93A mutation which is associated with familial ALS.

Figure J shows the effect of tacrolimus on yeast cells treated with hydrogen peroxide.

Figure K shows the effect of tacrolimus on gene expression in yeast cells treated with hydrogen peroxide.

Figure L shows the effect of tacrolimus on human cells on levels of SOD1 and SOD2 transcripts.

Figure M shows the effect of tacrolimus on the natural accumulation of endogenous aggregates of Tau and alpha-synuclein in aged mouse brain.

Figure N shows the effect of tacrolimus on phosphor-Tau and alpha-synuclein foci in the areas of the mouse brain.

Figure O shows the effect of tacrolimus and ascomycin on extending the lifespan of worms.

Figure P shows the tacrolimus and dihydrotacrolimus on extending the lifespan of worms.

Figure Q shows the effect of tacrolimus on the amphetamine induced rotational asymmetry assay at up to 6 months in rats which indicates promotion of neuronal regeneration.

Figure U shows the effect of tacrolimus in rat brains on alpha-synuclein and p-Tau which indicates effectiveness for treatment of neurodegenerational disorders such as Parkinson's disease and Alzheimer's disease.

Figure R shows the dose response range in worms treated with tacrolimus indicating greater effectiveness at lower doses.

Figure S shows the dose response of tacrolimus on Bus5 worms showing that lower doses are more effective than higher doses.

Figure T shows the extension on lifespan profile of S. Cerevisiae resulting from the administration of tacrolimus.

Figure V shows the extension on worm lifespan in Bu8 background caused by tacrolimus.

### DETAILED DESCRIPTION OF THE INVENTION

Accordingly the present invention provides a method of treating a disease characterised by protein aggregate deposition in neuronal cells which comprises administering to a human in need thereof not more than once a day an effective amount of tacrolimus or a close structural analogue thereof in a dose which does not cause immunosuppression and which produces a trough whole blood level of tacrolimus or its close structural analogue of at least 0.05 ng/m L.

Similarly, the present invention provides tacrolimus or a close structural analogue thereof for treating a disease characterised by protein aggregate deposition in neuronal cells, wherein tacrolimus or its close structural analogue is administered not more than once a day in a dose which does not cause immunosuppression and which produces a trough whole blood level of tacrolimus or its close structural analogue of at least 0.05 ng/mL.

Similarly, the present invention provides the use of tacrolimus or its close structural analogue in the manufacture of a medicament for the treatment of a disease characterised by the deposition of protein aggregates in neuronal cells which medicament contains an amount of tacrolimus or its close structural analogue that when administered once per day dose not cause immunosuppression and which has a trough whole blood level of at least 0.05 ng/mL.

The trough whole blood level may aptly be at least 0.075 ng/mL, for example at least 0.2 ng/mL, such as at least 0.3 ng/mL, or at least 0.1 ng/mL or at least 0.4 ng/mL.

In order to avoid immunosuppression the trough blood level will be less than one quarter that which is considered immunosuppressant when tacrolimus is used as an immunosuppressant. Generally, this means less than one third of the 4 ng/mL employed in order to prevent transplant rejection i.e. not more than 1.3 ng/mL.

It is believed that to benefit most from the therapeutic window offered by tacrolimus or its close structural analogue the whole blood trough level should be less than 1.2 ng/mL, for example less than 1.1 ng/mL such as 1.0 ng/mL.

Aptly the disease to be treated is ALS, Alzheimer's disease, Parkinson's disease, Huntington's disease and other syncleinopathies and taupathies such as Parkinson's disease dementia and frontotemporal lobe dementia and other dementia's and memory loss conditions which may be associated with age related increases with neurotoxic protein aggregation and/or increased oxidative stress or to defect of autophagy (the cells mechanism for removing damaged cellular components). Each of the above is individually disclosed herein for treatment by this invention. At present it is preferred that each of said diseases are treated using a very low dose of tacrolimus as described herein.

It is particularly advantageous for the treatment of such diseases that tacrolimus or its close structural analogue both relieves oxidative stress and promotes autophagy.

It is presently preferred to employ tacrolimus in this invention. However, it is also apt to employ a close structural analogue of tacrolimus. Such analogues retain the structure of tacrolimus from C₁ to C₁₇, C₁₉, C₂₀ and C₂₂ to C₃₄ but permit modifications at C₁₈ and/or C₂₁. Such modifications include permitting one hydrogen atom on either of C₁₈ or C₂₁, to be substituted by a C₁₋₄ alkyl, C₂₋₄ alkenyl, hydroxyl or methyoxyl group and also include replacing the C₂₁ propenyl group by a hydrogen atom, C₁₋₆ alkyl group, other C₂₋₆ alkenyl group, a hydroxyl group or a methoxyl group. Certain apt compounds include analogues of tacrolimus where the C₂₁ position is modified, for example C₂₁ propenyl group is replaced by a methyl group, an ethyl group or a propyl group. Other apt compounds include analogues where a C₁₈ hydrogen atom is replaced by a hydroxyl group. Certain of these compounds are less immunosuppressant than tacrolimus, for example the analogue wherein the C₂₁ propenyl group is replaced by a methyl group or the analogue wherein a C₁₈ hydrogen is replaced by a hydroxyl group (for example wherein the C₂₁ propenyl group is unchanged or replaced by a methyl, ethyl or propyl group). U.S. Pat. No. 5,376,663 discloses process for the preparation of analogues of tacrolimus. The C₂₁ propenyl group of tacrolimus may also be replaced by a fluoro C₁₋₆ alkyl group such as a 2-fluoroethyl group or by a different propenyl group such as a 1-methyl ethenyl group.

The term “does not cause immunosuppression” indicates that major side effects of immunosuppression do not normally occur. This results from a dose of tacrolimus or a close structural analogue being employed that does not substantially depress TNFα levels in the patient. It is believed that a dose of less than 1.3 mg per day of such compounds in a 70 kg adult (pro-rata for other body weights) may be considered not to lead to immunosuppression. However because individual personal variation the skilled person will be guided by the blood levels obtained as indicated hereinbefore.

Hence, the maximum daily dose of tacrolimus or a close structural analogue thereof that will be employed for a 70 kg patient will be 1.3 mg (pro-rata for other body weights) and more aptly a dose of not more than 1.0 mg and favourably of not more than 0.9 mg will be employed to a 70 kg patient, for example not more than 0.75 mg (pro-rata for other body weights) will be employed on any day give a wider separation between the desired effects and side effects.

Studies relating to conditions thought to involve the same underlying mechanism, indicate that a dose of not more than 0.6 mg per day, for example, not more than 0.5 mg per day such as 0.3 μg per day may be particularly apt as increasing doses can result in reduction in effectiveness in some treatments.

Studies suggest that a dose of not less than 0.05 mg per day of tacrolimus or a close structural analogue will be apt and a dose of not less than 0.1 mg per day may be favoured in some cases, for example a dose of not less than 0.15 mg.

Hence, apt daily doses for a 70 kg patient (with doses pro-rata for other weights) include 0.05 mg, 0.075 mg, 0.1 mg, 0.125 mg, 0.15 mg, 0.175 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5 mg, 0.525 mg, 0.55 mg, 0.575 mg, 0.6 mg and 0.625, 0.65, 0.675, 0.7, 0.725, 0.75, 0.775, 0.8, 0.825, 0.85, 0.85, 0.875, 0.9, 0.925, 0.95, 0.975, 1.0, 1.025, 1.075 and 1.2 of tacrolimus or a close structural analogue thereof (for example of tacrolimus). Doses of the active agent are aptly administered orally, for example as a discrete unit dose such as a tablet or capsule.

For convenience of provision of dosing a physician may advantageously wish to employ a fixed dose of tacrolimus across a patient group. Accordingly, provided herein are pharmaceutical compositions for use in treating a condition as described above (and in particular ALS, Parkinson's disease, Alzheimer's disease or Hodgkin's disease) which comprise 0.05 mg to 0.65 mg tacrolimus, such as 0.1 mg to 0.5 mg tacrolimus, for example 0.15 mg to 0.4 mg tacrolimus, such as 0.2 mg to 0.35 mg tacrolimus and in particular 0.3 mg tacrolimus. Such doses are aptly administered not more than once a day and preferably orally.

Because of the epigenetic mechanism of action of tacrolimus and its close structural analogues by which it provides its beneficial effects in treating disease characterised by protein aggregate deposition in neuronal cells, beneficial results may be obtained by dosing frequency of less than once per day, for example once every 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or more. Particularly suitable intervals for ease of patient use apart from daily may include on alternative days, once a week, once every 10 days, three times a month, once a fortnight or once a month.

From the preceding commentary on doses, the skilled person will understand that the present invention provides a method of treating a disease characterised by protein aggregate deposition in neuronal cells which comprises administering to a human in need thereof not more than once a day an effective amount of tacrolimus or a close structural analogue thereof wherein the dose is from 0.001 mg/kg to 0.02 mg/kg.

Similarly, the present invention provides tacrolimus or a close structural analogue thereof for treating a disease characterised by protein aggregate deposition in neuronal cells wherein tacrolimus or its close structural analogue is administered once a day at a dose of 0.001 mg/kg to 0.02 mg/kg.

Similarly the present invention provides the use of tacrolimus or a close structural analogue in the manufacture of a medicament for treating a disease characterised by protein aggregate deposition in neuronal cells wherein tacrolimus or its close structural analogue wherein the medicament contains 0.001 mg/kg to 0.02 mg/kg of tacrolimus or its close structural analogue.

Generally not more than 0.013 mg/kg, for example not more than 0.01 mg/kg will be employed. Aptly not more than 0.0085 mg/kg such as 0.007 mg/kg will be employed.

Generally more than 0.0014 mg/kg, for example more than 0.002 mg/kg will be employed.

It is presently considered particularly suitable to employ from 0.0014 mg/kg to 0.0085 mg/kg, for example from 0.002 mg/kg to 0.007 mg/kg of tacrolimus.

It will be appreciated that such doses are very different from doses employed in patients hereinbefore.

The present invention provides a method of treating a disease characterised by protein aggregate deposition in a neuronal cell which comprises administering to a human in need thereof by administration not more than once a day of an effective amount of tacrolimus or a close structural analogue to effect epigenetic modification which leads to an enhancement of autophagy and/or reduction in oxidative stress.

Similarly, the present invention provides tacrolimus or a close structural analogue thereof for use in the treatment of a disease characterised by protein aggregate deposition by administration not more than once a day of an amount which effects epigenetic modification which leads to an increase in autophagy and/or an improvement in oxidative stress.

Similarly, the present invention provides the use of tacrolimus or a close structural analogue thereof in the manufacture of a medicament for the treatment of a disease characterised by protein aggregate deposition in neuronal cells by administration not more than once per day which medicament contains an amount of tacrolimus or close structural analogue thereof which effects epigenetic modification which leads to an increase in autophagy and/or an improvement in oxidative stress.

The doses employed and the dose schedules may be as hereinbefore indicated.

In a further aspect the present invention provides a unit dose pharmaceutical composition containing 0.05 mg to 1.3 mg of tacrolimus or a close structural analogue thereof and a pharmaceutically acceptable carrier therefor for use in the treatment of a disease characterised by protein aggregate deposition in neuronal cells.

The disease may be as indicated hereinbefore, for example ALS.

The unit dose may contain not more than 1.2 mg, favourably not more than 0.75 mg, for example a dose of not more than 0.6 mg such as not more than 0.4 mg of tacrolimus or a close structural analogue thereof, preferably tacrolimus.

The unit dose may contain not less than 0.06 mg, favourably not less than 0.1 mg, for example not less than 0.15 mg of tacrolimus or a close structural analogue thereof, preferably tacrolimus.

The tacrolimus or its close structural analogue may be as solvates such as hydrates or alcoholates. Aptly tacrolimus is employed as a hydrate such as the monohydrate (when weights are referred to herein they do not include the weight of the solvating molecule).

Unit doses may contain any of the specific amounts set forth hereinbefore.

At present it is preferred that the unit dose will contain tacrolimus, for example as tacrolimus monohydrate.

If desired an existing commercial product such as Prograf® may be purchased and its contents divided to produce the desired dose which may then be placed into a hard gelatin capsule for oral administration.

The unit dose may be suitable for administration by injection but it is preferred that the unit dose is suitable for administration via the mouth. The unit dose may be liquid, for example a solution or suspension in a container, but it is considered preferable that the unit dose in non-liquid. Suitable solid unit dosage forms include tablets and capsules of which capsules are more apt. The skilled pharmaceutical chemist has decades of research into pharmaceutical compositions containing tacrolimus upon which to base preparation of unit doses.

In some cases, a pharmaceutical composition is formulated for oral administration. In some cases, the composition comprises a suspension of a compound in a suitable vehicle. Non-limiting examples of vehicles for oral administration include phosphate-buffered saline (PBS), 5% dextrose in water (D5W) and a syrup. The composition may be formulated to stabilize the consistency of a dose over a period of storage and administration. In some cases the composition may be a solution of the active compound dissolved in a diluent such as water, saline, and buffers optionally containing an acceptable solubilizing agent. In favoured form, the composition comprises a solid dosage form. In some cases, the solid dosage form comprises a capsule, a caplet, a lozenge, a sachet, or a tablet. In some cases, the solid dosage form is a liquid-filled dosage form. In some cases, the solid dosage form is a solid-filled dosage form. In some cases, the solid dosage form is a solid-filled tablet, capsule, or caplet. In some cases, the solid-filled dosage form is a powder-filled dosage form. In some cases, the solid dosage form comprises a compound in the form of micronized particles, granules or microcapsulated agent. In some cases, the composition comprises an emulsion which may contain a surfactant. In some cases, the solid dosage form comprises one or more of lactose, sorbitol, maltitol, mannitol, cornstarch, potato starch, microcrystalline cellulose, hydroxypropyl cellulose, acacia, gelatin, colloidal silicon dioxide, croscarmellose sodium, talc, magnesium stearate, stearic acid, pharmaceutically-acceptable excipients, colorants, diluents, buffering agents, moistening agents, preservatives, flavoring agents, carriers, and binders. In some cases, the solid dosage form comprises one or more materials that facilitate manufacturing, processing or stability of the solid dosage form or a flavoring agent.

When considering such formulation the skilled worker may call upon the many years experience in the art of formulating tacrolimus and of formulating low dose pharmaceuticals in general.

As indicated hereinbefore it is normal and greatly preferred not to administer the tacrolimus or close structural analogue more than once a day. However, in a much less preferred aspect, the present invention also provides methods and uses as hereinbefore defined modified so that tacrolimus or a close structural analogue is administered twice a day (as two divided doses).

Pharmacokinetic analysis carried out by the Applicant. A dose of 2 mg/kg/day in the mouse was found to correspond to a human oral dose 0.44-0.33 mg/day oral for a 70 kg person. The preferred dose from mouse studies of effects produced by tacrolimus indicated an oral dose of 0.22-0.16 mg/day for a 70 kg person with a blood level of 1-0.4 mg/mL as particularly effective.

In a further aspect the present invention provides a unit dose pharmaceutical composition containing 0.05 mg to 1.2 mg of tacrolimus or a close structural analogue thereof and a pharmaceutically acceptable carrier therefore for use in the treatment of a disease characterized by deposition of protein aggregates in neuronal cells.

Suitably the disease may be ALS. Suitably the disease may be Parkinson's disease. Suitably the disease may be Alzheimer's disease. Suitably the disease may be Huntingdon's disease.

The unit dose may contain not more than 0.9 mg or 0.75 mg, for example a dose of not more than 0.65 mg such as not more than 0.5 mg or not more than 0.45 mg of tacrolimus or a close structural analogue thereof, preferably tacrolimus.

Such unit dose may be for administration as described herein, particularly for oral administration.

The unit dose may contain not less than 0.05 mg, favourably not less than 0.1 mg, for example not less than 0.15 mg of tacrolimus or a close structural analogue thereof, preferably tacrolimus.

Hence, aptly the unit dose may contain from 0.05 mg to 0.9 mg, for example from 0.1 mg to 0.75 mg, for example from 0.15 mg to 0.6 mg or from 0.15 mg to 0.5 or 0.45 mg of tacrolimus. Such unit doses may be adapted for oral administration, for example as a tablet or preferably a capsule.

It is presently envisaged that the favoured daily doses of tacrolimus for the treatment of osteoporosis is from 0.05 to 0.65 mg, for example, 0.1 mg to 0.5 mg, such as 0.15 mg to 0.45 mg, for example, 0.3 mg. Such doses are aptly orally administered, and desirably not more than once per day for example, once a day using the unit doses described herein.

The Examples herein show that the effects of tacrolimus occurs in yeasts, worms and mammals indicating its ability to effect oxidative stress and autophagy are evolutionarily conserved. Hence, human disease will be treatable with tacrolimus (and close structural analogues) in the same way as with the other species including the mouse.

The following Examples illustrate the invention. Reference herein to RDC5 is to tacrolimus.

### EXAMPLE 1

### Demonstration of Clearance of Aggregates by Tacrolimus

Yeast expressing alpha-synuclein (A30P) were used to model aggregate build-up and its effect on viability. Yeast cultures were grown into stationary phase and aged (day 0). Viability measurements were made on subsequent days in strains expressing a variety of synuclein cassettes and under various treatments as shown in Figure A. The time taken (days) for the various cultures to reach 50% of their starting viability was calculated (median lifespan in Figure B) and is used as a general measure of the effect of an intervention on viability. The toxic effect of a-synuclein-A30P reduces the median life by over 50% (Figure B or by comparing the pcontrol (in Figure A) with the p-GFP-asyn-A30P (DMSO treated in Figure A)). Comparing the DMSO with the tacrolimus treated cultutes expressing the p-GFP-asyn-A30P cassette shows that RDC5 treatment is able to ameliorate the negative effect p-GFP-asyn-A30P expression has on the cultures.

Microscopic inspection of these cells showed that tacrolimus treatment was also able to reduce the number of cells with visible p-GFP-asyn-A30P aggregates as shown in Figure C.

### EXAMPLE 2

### Tacrolimus Ameliorates Polyq (HD) Toxicity

Tacrolimus was then tested in a number of worm models expressing aggregate prone proteins associated with a range of neurodegenerative diseases.

Worm (C. elegans) viability is assayed by synchronizing a culture and allowing it to age. Each worm in a given population in tested for viability by manually tapping it in the head and watching for movement. Repeating on a daily basis results in the determination of a viability (chronological lifespan) curve as shown below.

The aggregate prone poly Q-YFP protein is related to the pathogenic protein in Huntington's disease. When expressed in worms it shortens worm lifespan significantly (comparing black to blue curve). Tacrolimus is able to restore the lifespan profile of a worm expressing toxic polyQ proteins to a profile matching that of WT (see Figure D).

Tacrolimus treated worms were compared to their age matched controls microscopically to determine the mean aggregate peak intensity for each worm a measure of the extent to which aggregation is occurring in the two populations. The number and intensity of the polyQ aggregates in tacrolimus and control treated worms was also investigated (see Figure E).

Although there are no fewer peaks in tacrolimus treated worms, the peaks are of significant lower intensity indicating that their rate of formation is reduced or rate of dissolution is promoted (see Figure E).

### EXAMPLE 3

### Tacrolimus Ameliorates Alpha-Synuclein (PD) Toxicity

alpha-synuclein is the aggregates prone protein implicated in the pathology of Parkinson's disease. A-syn expressing worms have a shorter lifespan due to the toxicity of these aggregates. Tacrolimus is able to significantly improve the median lifespan of worms expressing toxic alpha-synuclein by 19%. This is seen in Figure F.

### EXAMPLE 4

### Tacrolimus Ameliorates SOD1 (ALS) Toxicity

Mutant SOD1 is the aggregate prone protein implicated in the pathology of certain types of ALS. Mutant SOD1 (127X) expressing worms have a shorter lifespan due to the toxicity of these aggregates and the subsequent increase in oxidative stress (comparing black to blue profiles). Tacrolimus is able to significantly improve the median lifespan by 37%, of worms expressing toxic aggregates of SOD1 (comparing black to red profiles). This is shown in Figure G.

### EXAMPLE 5

### Demonstration of Autophagy

Tacrolimus is seen to improve viability (lifespan) of SOD1 expressing C. elegans (seen in Example 4) but requires functional autophagy. bec-1 is an essential component of autophagy. Using RNAi knock down of bec-1 it has been shown that autophagy is required by tacrolimus for its lifespan extension and hence autophagy is part of the MOA for tacrolimus in reducing the toxicity of aggregates.

bec-1 encodes the C. elegans ortholog of mammalian autophagy proteins Atg6/Vps30/Beclin1; by homology, BEC-1 may be part of a Class III phosphatidylinositol 3-kinase complex that plays a role in localizing autophagy proteins to preautophagosomal structures.

As seen in Figure H, worms expressing SOD1 mutant (AM263) show a significant lifespan reduction compared to control worms (see previous section). Tacrolimus is able to reduce the toxic effect of the mutant and ameliorate the reduced viability phenotype.

RNAi knock down of bec-1 in a SOD1 mutant background reduces the lifespan still further (comparing yellow and orange curves). However, tacrolimus is unable to improve the lifespan profile (as it was previously) when bec-1 is knocked out (comparing orange with light grey curve).

This demonstrates that tacrolimus employs autophagy to improve the lifespan phenotype—indicating that autophagy is part of RDC5's mode of action (MOA).

### EXAMPLE 6

### Tacrolimus Ameliorates SOD1 Toxicity in Mammalian Cells

NSC-34 cells expressing SOD1-G93A (mutation known to cause familial ALS) and the control vector were treated with 100 ng/ml tacrolimus or control treated, for 24 hours.

Cells were then assessed for mortality using the inability to exclude Trypan blue. If a cell is unable to exclude Trypan blue from its cytoplasm it is classified as dead or dying. Therefore a lower the proportion of a population of cells which accumulate Trypan blue is indicative of fewer dead or dying cells.

As seen in Figure I, cell lines expressing WT SOD1 which have been control treated have approximately 15% of their cells which are unable to exclude trypan blue. This increases to 40% in cell lines expressing the pathogenic G93A mutant form of SOD1 indicating that a greater proportion of these cells are dead or dying. This confirms previous observations made in the literature that the pathogenic allele of SOD1 induces cell death.

When this cell line is treated with 100 ng/ml RDC5 the proportion of cells able to exclude trypan blue falls to 25% indicating that the fewer of the tacrolimus treated cells are dead or dying in comparison to their control treated equivalents.

### EXAMPLE 7

### Demonstration of Tacrolimus Effect on Oxidative Stress Resistance in Yeast and Hela Cells

Oxidative stress is also an important contributing factor in many neurodegenerative diseases. The following experiments were designed to show how tacrolimus can reduce oxidative stress and how it might be doing so.

Oxidative Stress Resistance in Yeast

3 day old yeast cultures treated as indicated were split into two, one treated with peroxide. Both cultures were assayed for viability. The data presented in Figure J is expressed as viability of peroxide treated culture divided by viability of non-peroxide treated culture for each group.

In summary, of the results shown in Figure J, tacrolimus treatment results in increase peroxide resistance (peroxide is used as a surrogate for oxidative stress) in 3 day old yeast cultures.

MOA for Oxidative Stress Resistance in Yeast

Transcript levels in yeast cells treated with control or tacrolimus were compared to reveal differences in gene expression that could lead to cells treated with RDC5 having improved resistance to oxidative stress.

The data shown in the two charts in Figure K compares gene expression levels in yeast under peroxide and RDC5 treatment. Tacrolimus induces transcription of a similar set of genes as treating with peroxide including super oxide dismutases.

This demonstrates that tacrolimus is promoting the cells natural mechanisms for dealing with oxidative stress.

Tacrolimus and Ox Stress in Human Cells

Hela cell gene expression patterns under RDC5 treatment were then also analysed to observe the expression of SOD1 and SOD2 gene expression under RDC5 or control treatment.

The results in Figure L show that Hela cells treated with tacrolimus have higher levels of SOD1 and SOD2 transcripts, suggesting that human cultured cells treated with tacrolimus have improved oxidative stress resistance as in yeast.

### EXAMPLE 8

### Mouse Model

MUS_ND_01

WT aged mice were used to study the natural accumulation of endogenous aggregates of Tau and a-synuclein in the aged mouse brain, which in humans are thought be involved in disease progression. The results are seen in Figure M.

Images of phospho-Tau and α-synuclein localization as determined by IHC were analysed computationally. Age induced increases in the intensity of both α-synuclein and phosphor-tau foci in the striatum can be reduced by treatment with 0.5 mg/kg/day tacrolimus. Although not age dependent, the intensity of phosphor-tau foci can be reduced in the substantia nigra by treatment with 0.5 mg/kg/day. Age induced increases in α-synuclein in the substantia nigra and phosphor-tau foci intensity in the thalamus cannot be reduced by treatment with any amount of tacrolimus tested here. α-synuclein foci intensity in the thalamus is neither affected by age nor tacrolimus treatment. This data suggests that tacrolimus treatment is both neuroactive and able to affect phospho-Tau and α-synuclein localization.

This demonstrates that 0.5 mg/kg/day tacrolimus had a superior effect to higher doses such as 8 mg/kg/day or 2 mg/kg/day in preventing accumulation of protein aggregates.

Data analysed by image_read.m programme with means and S.E.M. error bars plotted for the groups as indicated in Figure N. Significant deviation from 12 m Control (as assessed by p<0.05 t-test result) marked as shown (not significant unless indicated).

This again demonstrates the greater effect of 0.5 mg/kg/day tacrolimus dose than that at 2 mg/kg/day or 8 mg/kg/day.

### EXAMPLE 9

### Effects of Close Structural Analogues of Tacrolimus

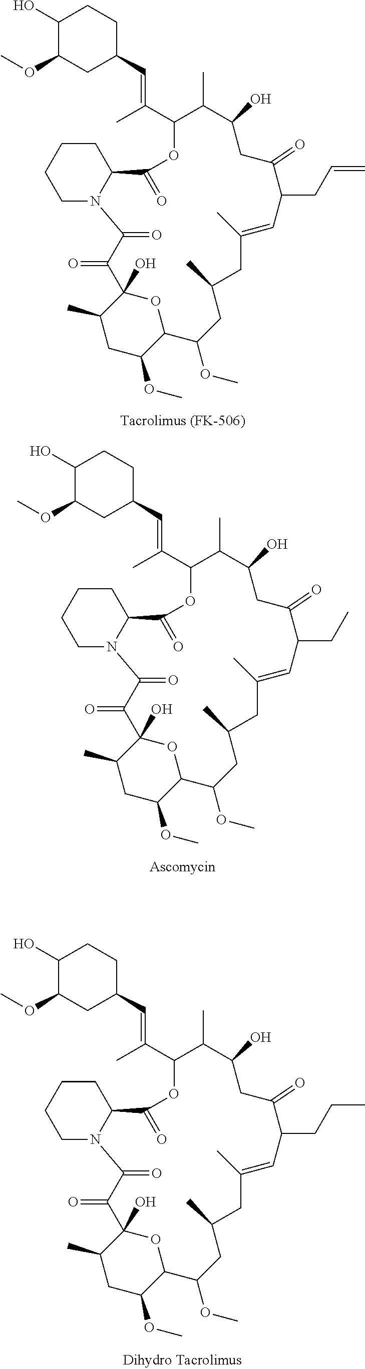

The lifespan plot shown in Figure O shows that Ascomycin (21-ethyl analogue) is able to extend the lifespan of worms as effectively as tacrolimus. This plot in Figure O also shows that different sources of tacrolimus have varied efficacy in extending lifespan in worms (Sigma vs Evita) possibly due to quality of source.

The lifespan plot in Figure P shows that Dihydrotacrolimus (21-propyl analogue) is able to extend the lifespan of worms almost as effectively as tacrolimus. This plot also shows that different sources of RDC5 have varied efficacy in extending lifespan in worms (Sigma vs Evita).

This data presented in Figures O and P demonstrates that the mechanisms by which very low dose tacrolimus exhibits its effects are also present in close structural analogues such as dihydrotacrolimus and the 21-ethyl analogue of tacrolimus.

### EXAMPLE 10

### Neuronal Regeneration

Summary of Trial

1 mg/kg/day RDC5 or vehicle was administered sub-cutaneously (s.c.) to 18 m.o female Fisher rats, 6 days a week starting 3 weeks after 6-OHDA infusion and continued until the end-point (approx. 6 months). The behavioural impairment of rats was evaluated by amphetamine induced rotation asymmetry test started from 2 weeks after 6-OHDA lesioning and continued every 2 months until the end of follow-up period (approx. 6 month). HPLC was used on neural samples to determine levels of dopamine and its metabolites post-mortem. Histology of brain slices from the un-lesioned side of the brain was used to investigate the age-related accumulation of p-Tau and alpha-synuclein as well as the dopaminergic neuron cell counts. 3 month cull n=5, 6 month cull n=20.

Rotational Asymmetry Assay

In one study, aged rats were asymmetrically lesioned with 6-OHDA, and assessed behaviourally by the amphetamine induced rotational asymmetry assay, 2 weeks and 2, 4 and 6 months after lesioning. In lessioned animals there is a tendency to spontaneously rotate towards the side of the lesion (i.e. Clockwise rotations minus Counter Clockwise rotations (CW-CCW) is positive when lesioned on the left side of the brain). This is known as ipsilateral movement. When the effect of lesioning is reduced through treatment with a neuro-regenerative compound, CW-CCW should be closer to 0 than control treated age-matched controls i.e. the extent of ipsilateral movement decreases.

In this study, as shown in Figure Q, both groups of rats (control and RDC5 treated) were assayed prior to treatment (15 days) and showed no significant difference in spontaneous rotation with CW-CCW being approximately 2500. This indicates that there is no difference between the two groups prior to treatment. When the assay was repeated at the 2, 4 and 6 month time-points the difference between the control and RDC5 treated groups became progressively larger, with the RDC5 treated rats showing progressively less ipsilateral rotation. In contrast, control treated rats show no significant decrease in ipsilateral movement. The result of this trend is a significant reduction in ipsilateral movement in RDC5 treated rats, relative to controls by the 6 month timepoint. This suggests RDC5 is able to promote neuro-regeneration.

IHC Analysis of p-tau and α-synuclein

IHC was used to assess the fraction of cells containing α-synuclein and phospho-Tau aggregates as well as the pattern of the aggregation in brain slices from the rats. The results are shown in Figure U.

Data are presented as mean+SEM. Vehicle 3 month, n=3; Vehicle 6 month, n=6; RDC5 1 mg/kg 3 month, n=5; RDC5 1 mg/kg 6 month, n=5. Data was analyzed using Student's t-test vehicle vs. RDC5.

As seen in Figure U, RDC5 1mg/kg 3 months group presented significantly lower counts of both p-Tau and α-synuclein positive cells than control treatment.

This data is consistent with RDC5 being beneficial for the treatment of aggregate based neurodegenerational disorders such as Parkinson's disease and Alzheimer's disease.

Studies (staining with tyrosine hydroylase) indicated that the effect was not produced by increasing dopamine levels.

### EXAMPLE 11

Unit Dose

A hard gelatine capsule was filled with the following composition:

| | Formulation | | Reference |
|---|---|---|---|
| Component | (%) | Function | Standard |
| RDC5 | 0.30 | Active ingredient | USP |
| Lactose monohydrate | 89.45 | Diluent | Ph Eur |
| Hydroxypropylmethyl | 6.00 | Binder | Ph Eur |
| cellulose | | | |
| Croscarmellose sodium | 4.00 | Super-disintegrant | Ph Eur |
| Magnesium stearate | 0.25 | Lubricant | Ph Eur |
| Ethanol | qs | Binder fluid | |

One capsule as above containing 0.3 mg tacrolimus was administered daily to (number) healthy human volunteers for three successive days. No significant changes in blood TNF-α levels occurred as a result of the administration. Similarly, no change in TNF-α levels occurred as a result of administering 0.6 mg daily of tacrolimus to healthy volunteers. The average trough level of tacrolimus (level after 24 hours of administration of each dose) observed was approximately 220 pg/mL. The average peak level of tacrolimus observed was approximately 3700 pg/mL and the average area under the curve was approximately AUC O_t=23500 (h*pg/mL).

The above capsules may be used to provide the treatments described herein before.

Use of two such capsules simultaneously to provide a single dose of 0.6 would be expected to result in a trough level of about 440 pg/mL.

### EXAMPLE 12

Dose Optimization for Tacrolimus

The facts outlined below shows that RDC5 produces as bell shaped dose response in: worm lifespan extension, response to oxidative stress of Osteoblast cells.

These dose define the efficacious dose range for RDC5.

| | | | % | | % | | % |
|---|---|---|---|---|---|---|---|
| | | | increase | | increase | | increase |
| | Control | RDC5 | wrt | RDC5 | wrt | RDC5 | wrt |
| Bus5 | Treatment | 1 ug/ml | control | 10 ug/ml | control | 40 ug/ml | control |
| Healthspan | 10.93 | 14.55 | 33 | 13.43 | 23 | 10.28 | −6 |
| Median | 15.08 | 18.54 | 23 | 19.03 | 26 | 15.17 | 0.5 |
| Lifespan | | | | | | | |

Lower doses of 1ug/ml and 10 ug/ml are most efficacious at extending CLS in Bus5 worms. (20 ug/ml contaminated)

| | | | % | | % | | % | | % |
|---|---|---|---|---|---|---|---|---|---|
| | | | increase | | increase | | increase | | increase |
| | Control | RDC5 | wrt | RDC5 | wrt | RDC5 | wrt | RDC5 | wrt |
| Bus8 | Treatment | 1 ug/ml | control | 10 ug/ml | control | 20 ug/ml | control | 40 ug/ml | control |
| Healthspan | 6.87 | 6.90 | 0.5 | 7.85 | 14 | 6.23 | −9 | 6.67 | −3 |
| Median | 10.10 | 10.86 | 7.5 | 12.18 | 20 | 11.70 | 16 | 11.70 | 16 |
| Lifespan | | | | | | | | | |

Low dose of 10 ug/ml is the most efficacious at extending Healthspan in Bus8 worms. Higher doses of 20 ug/ml and 40 ug/ml match the efficacy of 10 ug/ml at Median and Maximal Lifespan extension but not Healthspan.

(Coli food source and FUDR have been ruled out as possibly affecting lifespan.)

As shown in Figure R the dose response in worms→narrow window of efficacy in CLS at lower end of doses tested, 10 ug/ml is consistently the most efficacious dose for improving healthspan, even lower doses are sometime also effective and high dose of 40 ug/ml is always the least effective.

In Bus8 worms a small efficacious dose range for Healthspan, larger range for dose response when considering Median Lifespan, but a efficacy peaking at 10 ug/ml for both Healthspan and Median Lifespan extension. This is shown in Figure S.

In Bus8 worms again there is a small efficacious dose range for Healthspan and a larger range for dose response for Median Lifespan. Efficacy peaking at 1 ug/ml for Healthspan extension and 10 ug/ml for Median Lifespan extension.

In further experiments it was determined that dosing on two thirds of days also produced significant increases in lifespan compared to control and that dosing on one third of days produced significant increases in lifespan (except for when dosing was not commenced early in lifespan). This is consistent with the desired therapeutic effects described herein being achieved with less than every day administration.

### EXAMPLE 13

### Tacrolimus Delays Ageing in Yeast and Worms

The yeast bioscreen measures the Chronological Lifespan Profile of an ageing yeast cultures. The median lifespan is defined as the time at which 50% of a given population has died. Relative viability (expressed in the below chart) compares the effect of treatment on a cultures viability as a fraction of control vehicle treated cultures. RDC5 was identified as being able to extend the chronological lifespan profile of S. Cerevisiae without effecting growth rate

RDC5 treated yeast cultures have a significantly longer median lifespan than control treated cultures. RDC5 is thus believed to be able to delay ageing and the onset of age-related diseases such as unwanted fat accumulation. The effect on life span is shown in Figure T.

Worm cultures of at least 80 worms were manually assessed daily for the duration of the lifespan. RDC5 is also able to extend the Healthspan and Median lifespan of populations of worms as well as yeast.

Effect of Tacrolimus on Worm Lifespan in Bus8 Background is shown in Figure V.

The low dose of 10 ug/ml is the most efficacious at extending Healthspan in Bus8 worms.

1-37. (canceled)

38. A pharmaceutical composition comprising Tacrolimus or a close structural analogue thereof wherein tacrolimus or its close structural analogue is administered not more than once a day in a dose which does not cause immunosuppression and which produces a trough whole blood level of tacrolimus or its close structural analogue of at least 0.05 ng/mL, wherein the Tacrolimus or a close structural analogue is in a therapeutically effective amount for treating one or more conditions selected from the group consisting of:
a disease characterised by protein aggregate deposition in neuronal cells;
amyotrophic lateral sclerosis;
Alzheimer's disease;
Parkinson's disease;
Huntingdon's disease;
Sycleinopathy;
Taupathy;
Dementia; and
combinations thereof.

39. The pharmaceutical composition of claim 38 wherein the trough whole blood level produced is at least 0.075 ng/mL, at least 0.2 ng/mL or at least 0.3 ng/mL.

40. The pharmaceutical composition of claim 38 wherein the trough whole blood level produced is less than 1.2 ng/mL, less than 1.1 ng/mL or less than 1.0 ng/mL.

41. The pharmaceutical composition of claim 38 which comprises tacrolimus.

42. A pharmaceutical composition comprising Tacrolimus or a close structural analogue thereof wherein tacrolimus or its close structural analogue is administered once a day at a dose of 0.001 mg/kg to 0.02 mg/kg wherein the Tacrolimus or a close structural analogue is in a therapeutically effective amount for treating one or more conditions selected from the group consisting of:
a disease characterised by protein aggregate deposition in neuronal cells;
amyotrophic lateral sclerosis;
Alzheimer's disease;
Parkinson's disease;
Huntingdon's disease;
Sycleinopathy;
Taupathy;
Dementia; and
combinations thereof.

43. The pharmaceutical composition of claim 42 wherein the dose is 0.001 mg/kg to 0.02 mg/kg.

44. The pharmaceutical composition of claim 42 wherein the dose is not more than 0.013 mg/kg.

45. The pharmaceutical composition of claim 42 wherein the dose is not more than 0.01 mg/kg, 0.0085 mg/kg or 0.007 mg/kg.

46. The pharmaceutical composition of claim 42 wherein the dose is more than 0.0014 mg/kg or 0.002 mg/kg.

47. The pharmaceutical composition of claim 42 wherein the dose is 0.0014 mg/kg to 0.0085 mg/kg or 0.002 mg/kg to 0.007 mg/kg.

48. The pharmaceutical composition of claim 42 which comprises tacrolimus.

49. A unit dose pharmaceutical composition which comprises 0.05 mg to 1.3 mg of tacrolimus or a close structural analogue thereof and a pharmaceutically acceptable carrier therefor wherein the unit dose pharmaceutical composition is a therapeutically effective amount for treating one or more conditions selected from the group consisting of:
a disease characterised by protein aggregate deposition in neuronal cells;
amyotrophic lateral sclerosis;
Alzheimer's disease;
Parkinson's disease;
Huntingdon's disease;
Sycleinopathy;
Taupathy;
Dementia; and
combinations thereof.

50. The unit dose pharmaceutical composition of claim 49 which is an orally administrable dosage form.

51. The unit dose pharmaceutical composition of claim 49 which does not contain more than 1.2 mg, 0.75 mg, 0.6 mg or 0.4 mg of tacrolimus or a close structural analogue thereof.

52. The unit dose pharmaceutical composition of claim 49 which comprises not less than 0.06, 0.1 or 0.15 mg.

53. The unit dose pharmaceutical composition of claim 49 adapted for administration by mouth.

54. The unit dose of claim 49 wherein the disease is ALS, Parkinson's disease, Alzheimer's disease, Huntingdon's disease.

55. The unit dose of claim 49 which comprises tacrolimus.

56. The unit dose of claim 49 which comprises not more than 0.9 mg, 0.75 mg, 0.65 mg or not more than 0.5 mg or not more than 0.45 mg of tacrolimus.

57. The unit dose of claim 49 which comprises not less than 0.05 mg, not less than 0.1 mg or not less than 0.15 mg of tacrolimus or close structural analogue thereof.

58. The unit dose of claims 49 in the form of a tablet or capsule.

59. A method of treating a disease in a patient in need thereof which comprises administering to a patient in need thereof not more than once a day an effective amount of tacrolimus or a close structural analogue thereof in a dose which does not cause immunosuppression and which produces a trough whole blood level of tacrolimus or its close structural analogue of at least 0.05 ng/mL, wherein the Tacrolimus or a close structural analogue is in a therapeutically effective amount for treating one or more conditions selected from the group consisting of:
a disease characterised by protein aggregate deposition in neuronal cells;
amyotrophic lateral sclerosis;
Alzheimer's disease;
Parkinson's disease;
Huntingdon's disease;
Sycleinopathy;
taupathy;
dementia; and
combinations thereof.

60. The method of claim 59 wherein the trough whole blood level is at least 0.075 ng/mL, at least 0.2 ng/mL or at least 0.3 ng/mL.

61. The method of claim 59 wherein the trough whole blood level is less than 1.2 ng/mL, less than 1.1 ng/mL or less than 1.0 ng/mL.

62. The method of claim 59 which employs tacrolimus.

63. A method of treating a disease in a patient which comprises administering to a patient in need thereof not more than once a day an effective amount of tacrolimus or a close structural analogue thereof wherein the dose is from 0.001 mg/kg to 0.02 mg/kg, wherein the Tacrolimus or a close structural analogue is in a therapeutically effective amount for treating one or more conditions selected from the group consisting of:
a disease characterised by protein aggregate deposition in neuronal cells;
amyotrophic lateral sclerosis;
Alzheimer's disease;
Parkinson's disease;
Huntingdon's disease;
Sycleinopathy;
taupathy;
dementia; and
combinations thereof.

64. The method of claim 63 wherein the dose is 0.001 mg/kg to 0.02 mg/kg.

65. The method of claim 63 wherein the dose is not more than 0.013 mg/kg, not more than 0.01 mg/kg, 0.0085 mg/kg or 0.007 mg/kg.

66. The method of claim 63 wherein the dose is more than 0.0014 mg/kg or 0.002 mg/kg.

67. The method of claim 63 wherein the dose is 0.0014 mg/kg to 0.0085 mg/kg or 0.002 mg/kg to 0.007 mg/kg.

68. The method of claim 63 wherein the dose is tacrolimus.

69. The method of claim 63 wherein the condition is Parkinson's disease dementia, frontotemporal lobe dementia, other dementia's and memory loss conditions associated with eye related increase of neurotoxic protein aggregation and/or increased oxidative stress, or to a defect in autophagy

70. A method of treating a disease characterised by protein aggregate deposition in a neuronal cell which comprises administering to a patient in need thereof by administration not more than once a day of an effective amount of tacrolimus or a close structural analogue to effect epigenetic modification which leads to an enhancement of autophagy and/or reduction in oxidative stress.

71. The method of claim 70 which employs tacrolimus.
